(19) United States
(12) Patent Application Publication
(10) Pub. No.: US 2008/0233184 A1
(43) Pub. Date: Sep. 25, 2008

(54) Combination enzyme products

(76) Inventor: Kenneth Looney, Houston, TX (US)

Correspondence Address: Joseph P. Kincart, Esq., 8153 Middle Fork Way, Jacksonville, FL, 32256, US

(21) Appl. No.: 11/891,824

(22) Filed: Aug. 13, 2007

Related U.S. Application Data
(60) Provisional application No. 60/918,879, filed on Mar. 19, 2007.

(51) Int. Cl. A61K 9/48

(52) U.S. Cl. 424/451; 424/456

(57) ABSTRACT

The present invention provides a unique package as well as methods packaging an enzyme portion and a substance portion. The package is bifurcated to maintain an enzyme separate from a primary substance until a predetermined set of environmental conditions is met. When the environmental conditions are met, the enzyme acts as a catalyst of the primary substance facilitating a change of the primary substance into a second substance. The environmental conditions can include, for example, conditions associated with a human alimentary canal.

### CROSS REFERENCE TO RELATED APPLICATIONS

The present application claims priority to pending patent application S/N 60/918,879 filed, Mar. 19, 2007 and entitled, “Combination Enzyme Products,” the contents of which are relied upon and incorporated by reference

### FIELD OF THE INVENTION

The present invention relates to bifurcated packages isolating an enzyme from a subject matter. More specifically, the present invention relates to a combination package and methods and apparatus for manufacturing the combination package, the combination package containing an enzyme and a first subject matter, each isolated from each other. Degradation of the package integrity permits the enzyme to act as a catalyst for a first subject matter to create one or more second subject matters.

### BACKGROUND OF THE INVENTION

Enzymes are essential to human life. A human body includes approximately ten trillion cells. Each cell performs one or more functions, which enable a human body to live. Work being done inside a cell to perform a particular function is accomplished via enzymes. An enzyme acts as a catalyst in a cell by enabling the cell to carry out a chemical reaction efficiently. Such reactions facilitate the cell in building, or taking apart, various biological components according to a specific need. Which function an enzyme facilitates is based upon the make up of the enzyme.

Essentially, enzymes are proteins made from amino acids. When an enzyme is formed, it is made by stringing together and folding a chain of approximately 100 to 1,000 amino acids in a specific sequence. The characteristics of a particular chain determine whether the enzyme is suitable as a catalyst for a specific chemical reaction, and thereby a particular function.

Although enzyme biochemistry has been the subject of intensive study the role of enzymes in supplementation and therapy is not widespread knowledge. Most people have little knowledge of which enzyme will work with which substances to obtain a desired result. Consequently, most, if not all human beings due not operate, as a biological machine, at their optimum capacity. Enzymes carry a vital energy factor needed for every chemical action and reaction that occurs in our body. However, the approximately 2000 different enzymes found in the human cell presents a considerable task to fully understand. Add to this number the combination of co-enzymes for nearly 100,000 or so various chemicals which enable us to see, hear, feel, move, digest food and think; and it is understandable why an average person does not command a thorough understanding of enzymes at work in their body.

Similarly, although a health care practitioner may be knowledgeable as to which enzyme may promote a particular cellular activity, such as digestion and assimilation of a particular food source, an average health care practitioner may not have the time or motivation to specifically associate one or more particular enzymes with a particular substance for which the enzyme will act as a catalyst.

Therefore it would be useful to have a method and mechanism to provide a particular enzyme to a person at a particular time based upon a need the person has at that time.

### SUMMARY

Accordingly, the present invention provides bifurcated packages for providing an enzyme portion and a primary substance isolated from each other for as long as the integrity of the package is maintained. According to the present invention, the package maintains isolation of the enzyme from the primary substance sufficient to prevent the enzyme from acting as a catalyst in a chemical reaction which changes the primary substance to one or more secondary substances.

In some preferred embodiments, the package includes a softgel containing an omega-3 oil or an omega-6 oil as a primary substance, surrounded by a digestive enzyme contained within a capsule. The softgel isolates the omega-3 from the omega-3 oil thereby preventing pre-digestion of the omega-3 oil. The capsule provides a convenient package containing a predetermined digestive enzyme appropriate for use with the omega-3 oil. In addition, the capsule provides predetermined amounts of both digestive enzyme and the omega-3 oil, thereby eliminating uncertainties by a consumer of the product. Upon ingestion by a human being or other mammal, the capsule and the softgel breakdown according to normal digestive processes thereby combining the enzyme and the primary substance. The combination of the enzyme and the omega-3 oil allows the enzyme to act as a catalyst to create a secondary substance from the omega-3 oil.

### BRIEF DESCRIPTION OF THE DRAWINGS

As presented herein, various embodiments of the present invention will be described, followed by some specific examples of various components that can be utilized to implement the embodiments. The following drawings facilitate the description of some embodiments:

FIG. 1 illustrates an embodiment of a package separating an enzyme from a primary substance.

FIG. 2 illustrates an additional embodiment of a package separating an enzyme from a primary substance, wherein the enzyme portion is generally concentric and within the primary substance.

FIG. 3 illustrates some embodiments wherein at least one of the enzyme and the primary substance includes a cylindrical shape.

FIG. 4 illustrates some embodiments of the present invention with an outer boundary and a primary substance portion isolated from an enzyme portion via micro-encapsulation.

### DETAILED DESCRIPTION OF PREFERRED EMBODIMENTS

The present invention provides methods and devices for providing a package with a first portion containing an enzyme and a second portion containing a primary substance, wherein during a time period in which the integrity of the package is maintained, the enzyme is prevented from acting as a catalyst to the primary substance or facilitate hydrolyzing the primary substance. In addition, when the integrity of the package is disrupted, the enzyme acts as a catalyst in a chemical reaction which changes at least a portion of the primary substance into a second substance.

Embodiments can include a particular enzyme packaged with a specific primary substance in an amount, and in an environment, likely to promote a desired chemical reaction when the enzyme comes into contact with the primary substance.

Packages

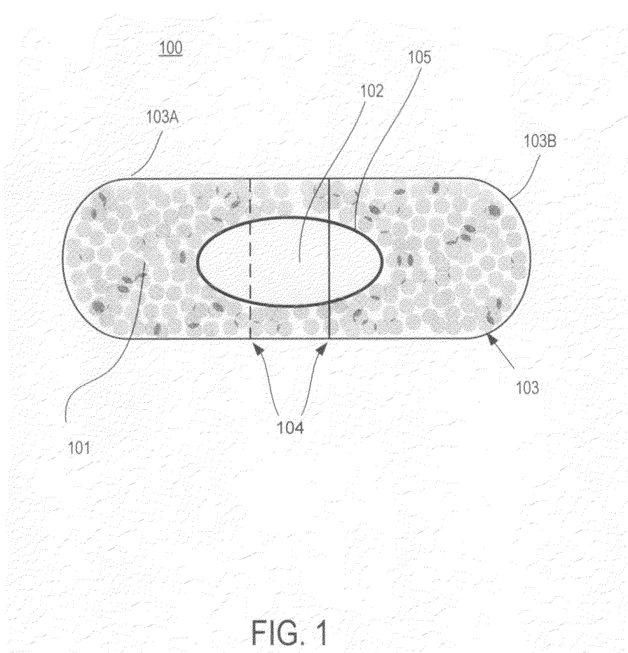

Referring now to FIG. 1, some preferred embodiments of the present invention are illustrated. As illustrated, a capsule 100 includes an enzyme portion 101 in addition to a primary substance 102. Separation of the enzyme portion 101 and the primary substance 102 is maintained so long as the integrity of the package is maintained. The enzyme portion 101 includes one or more enzymes, or a composition including an enzyme and other substance for which the enzyme does not act as a catalyst in its packaged state. The enzyme portion 101 can be in a powdered state, or any other state that can be accessed by a human alimentary canal, such as, for example, micro-encapsulated or gel.

Generally, the enzyme portion is maintained in a separated state from the primary substance 102 while the integrity of the package is maintained by including an outer boundary 103 and an inner boundary 105. The inner boundary 105 includes a material effective at isolating the enzyme portion from the primary substance portion while the integrity of the inner boundary is intact and while the enzyme portion is placed on one side of the boundary and the primary substance is placed on another side of the inner boundary. The outer boundary 103 defines an outer containment area sufficient to contain one of the enzyme portion and the primary substance portion and the inner boundary. The outer boundary 103 also includes a material effective at containing the enzyme portion, the inner boundary and the primary substance portion

As illustrated, some embodiments can include an enzyme portion 101 contained in an outer boundary 103. The outer boundary 103 can include a two part capsule formed from two capsule halves 103A-103B. Each half 103A and 103B includes an open end 104 which can be brought together to form a seal to prevent the enzyme from spilling outside of the capsule 100. The size of a capsule can determine an amount of enzyme that will be contained by the capsule. The amount of enzyme contained by the capsule 103A-103B will also vary depending upon a volume occupied by the primary substance 102 and a inner boundary 105.

Capsules can be formed from any substance which can contain the enzyme and also allow for the integrity of the capsule to be disrupted when the capsule is exposed to a predetermined condition. Capsules meant for human consumption can therefore be fashioned, by way of example, from one or more of: gelatin, including one or both of hard and soft gelatin; vegetable base; water-soluble plastic, such as certain varieties of polyvinyl alcohol (PVOH); pullulan; Npcapsules; starch; ach chitosan. Capsule size can be according to standard sizes 000 to 3 or a custom size suitable for a particular application. Other containers may include, gel tabs, composites, tablets, pills or other packaging combinations which maintain a bifurcated state until a predetermined set of conditions is met.

In addition to the enzyme portion 101 contained within the capsule, the capsule will also contain an inner boundary 105 encasing a primary substance 102. The primary substance 102 is preferably matched with the enzyme portion 101 so that when the integrity of the inner boundary 105 encasing the primary substance 101 is disrupted, the primary substance 102 comes into contact with the enzyme portion 101 and the enzyme acts as a catalyst in a chemical reaction which causes at least some of the primary substance to be changed into a second substance (not illustrated). The inner package can include a soft gel encapsulation or packaging or capsule, such as those described above.

Other embodiments can include, for example, a hard outer capsule 103 and a soft inner boundary 105; a hard outer boundary 103 and a hard inner boundary 105; a hard outer capsule and an inner caplet; a hard outer package and a inner tablet. Still other embodiments can include a outer soft gel encapsulation that surrounds an inner hard capsule or inner hard caplet or tablet.

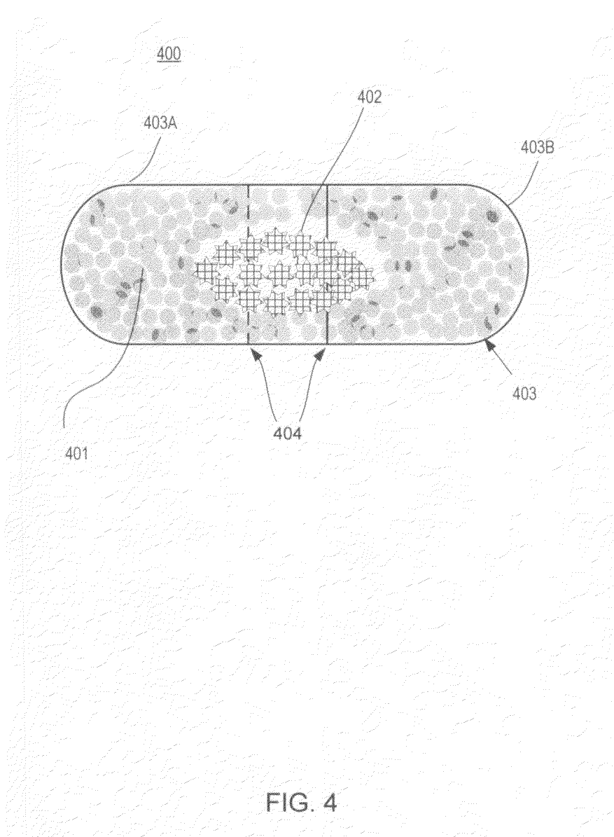

Referring now to FIG. 4, in still other embodiments, a capsule 400 can include a hard outer capsule 403 and contain an enzyme portion 401 including one or more enzymes or enzyme composition. The enzyme portion can such as, in powder form, or any other state that can be accessed by a human alimentary canal, such as, for example, micro-encapsulated or gel.

As discussed above with reference to FIG. 1, in some embodiments, the outer capsule 403 can include a two part capsule formed from two capsule halves 403A-403B. Each half 403A and 403B can include an open end 404 which can be brought together to form a seal with the other capsule half 403A-403B, and thereby prevent the enzyme portion 101 from spilling outside of the capsule 400. The size of a capsule can determine an amount of enzyme that will be contained by the capsule. The amount of enzyme contained by the capsule will also vary depending upon a volume occupied by the primary substance 402 and a primary substance 402. The primary substance 402 can include any substance for which the enzyme portion 401 can act as a catalyst when it come into contact with the primary substance 402.

As illustrated in FIG. 4, second substance 402 can be micro encapsulated or otherwise contained in small amounts wherein the second substance 402 is separated from the enzyme portion while the integrity of the encapsulation is maintained. Some examples can include microcapsules of the second substance 402 and microbeads of the second substance 402.

Some preferred embodiments include package 105 that is a softgel single piece encapsulation encasing a primary substance that is an omega-3 oil, such as, for example fish oil or flax seed oil. The enzyme can include an enzyme which will act as a catalyst to help digest the omega-3 oil when the capsule is ingested into a human body.

The enzyme portion 101 is able to act as a catalyst when the human body's natural digestive processes breakdown the capsule 100 thereby releasing the enzyme portion 101 into the stomach, and also breaks down the softgel package 105 thereby also releasing the omega-3 oil into the stomach. The enzyme portion 101 is immediately present to the omega-3 oil in an amount, and in an environment, conducive to acting as a catalyst in a chemical reaction which digests the omega-3 oil. As the omega-3 oil is digested, it is changed into a second substance different than omega-3 oil.

In some preferred embodiments, an enzyme portion 101 that is packaged with primary substance to aid digestion can include one or more of: protease, amylase, lipase, cellulose, lactase, maltase, and sucrase. An enzyme portion can include, for example: one or more of: between about 30,000 HUT and 180,000 HUT of protease; between about 6,000 and 80,000 DU of amylase; between about 200 LU and 12.00 LU of lipase; and between about 20 and 4000 CU of cellulase.

In another aspect, some or all of an enzyme portion 101 can be included in a capsule 100 which will compliment a benefit provided by a primary substance 102. In such embodiments, a capsule can be assembled which provides a predetermined benefit from the combination of the enzyme and the primary substance and still maintains a separation of the enzyme 101 and the primary substance 102 until after the integrity of the package 105 is broken.

Prepackaging of an enzyme and a primary substance 102, such as the omega-3 also provides for a convenient method of administering a predetermined amount of both the enzyme 101 and the primary substance 102. In addition, because the capsule releases the enzymes into the stomach first, the stomach can be fully prepared to receive the omega-3 oil by the time the digestive processes are able to break down the softgel package 105. In some embodiments, a thickness of the softgel encasing 105 and its propensity to be broken down by the human digestive system can be predetermined to correlate with a desired time interval between the release of the enzyme into the alimentary canal, such as, in the stomach or small intestine or the large intestine and the time the primary substance is released into the stomach.

Other embodiments can include, by way of non-limiting examples: a) an enzyme portion including lipase and a primary substance including an essential fatty acid, such as one or both of omega 3 and omega 6 fatty acids and a carrier oil, such as, for example, safflower oil or flaxseed oil or other medium; b) an essential fatty acid in powder form in one or the other of the outer package and the inner package, and one or more of lipase, amylase and protease in a complimenting package (i.e. outer capsule and inner capsule); and c) one or both of protease and cellulase in one or the other of the outer package and the inner package, and a microencapsulated probiotic in a complimenting package (i.e. outer capsule and inner capsule).

In another aspect, a primary substance, such as, for example a nutraceutical, can be packaged according to the present invention is a separate state from an enzyme portion in a package designed to break down at a predetermined portion of the human digestive tract. The package may break down for example, under conditions of a particular pH or acid environment.

Enzymes packed with the primary substance, but separate from the primary substance would come into contact with the primary substance when the packaging was broken down by conditions encountered in the human digestive tract and hydrolyze the primary substance. The hydrolyzation of the primary substance facilitates the bioavailability for absorption into the body. For example most fatty acids are presented as triglycerides, and therefore need a lipase to release the free fatty acids for absorption. The enzyme lipase in one capsule portion or other package, ensures the hydrolysis of the triglyceride containing the omega fatty acid and its bioavailability upon degradation of the package.

For these purposes a nutraceutical can include a food substance or chemical with a medicinal effect on human health, such as those listed by the U.S. Food and Drug Administration or others with medicinal claims. Some examples can include: flavanoids, anti oxidants; gamma linolenic acid, beta carateins, anthocyanins; botanical and herbal extracts; Ginseng; ginko bilboa and garlic.

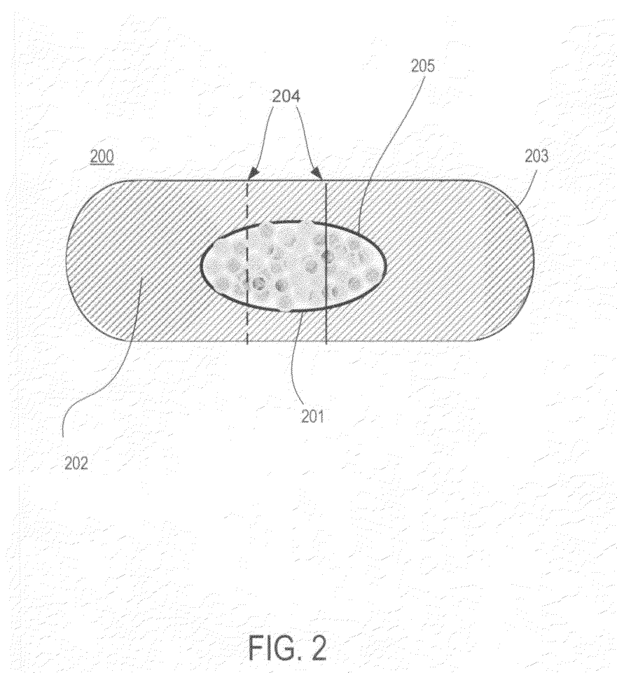

Referring now to FIG. 2, in some embodiments, an enzyme 201 can be packaged internally to the primary substance 202, such that the primary substance 202 surrounds the enzyme, essentially opposite to the scenario described in FIG. 1.

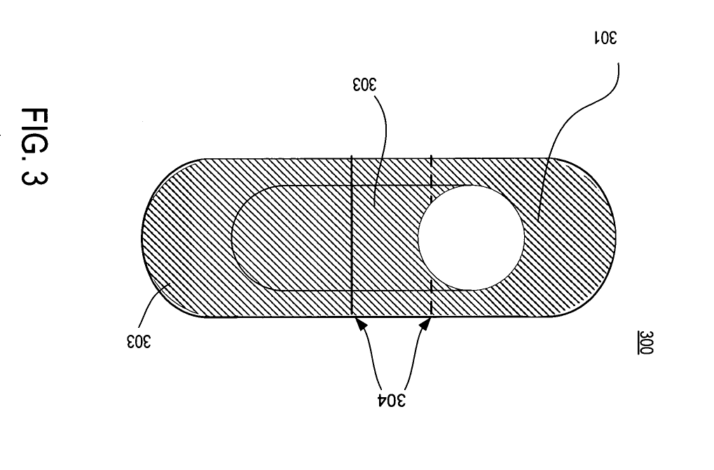

Referring now to FIG. 3, common packaging practices provide for generally oval shaped softgel tabs, with common capsules available in sizes 000 to 3. However the present invention can incorporate additional shapes and sizes. Accordingly, some embodiments can include an interior sealed portion 303 can be generally cylindrical with flat ends. The cylindrical shape with flat ends can be conducive to automated handling. Automated assembly equipment may more readily be able to handle a capsule with one or more portions which are flat. Still other shapes may include a caplet or tablet shape.

In still other embodiments, microencapsulation technologies can be employed to seal multiple discrete portions of one or both of an enzyme and a primary substance. The microencapsulation will isolate each of the enzyme and the primary substance until some event causes the integrity of the microencapsulation to be voided. The event may include, for example, ingestion of the microencapsulated enzyme or primary substance.

It should be noted, that although the examples described above generally referred to one enzyme and one primary substance, compounds of multiple enzymes and multiple primary substances are also within the scope of the present invention. Accordingly, some embodiments may include multiple softgels contained within a capsule, each capsule encasing one or more enzyme or primary substance. In addition, those embodiments employing microencapsulation can include any combination of enzymes and primary substances desired, each individually encased within a microencapsulation, or encased as a composition within microencapsulation.

In some specific embodiments, a enzyme portion can be combined with a primary substance in one or both of: an amount of one or both and a ratio of one to the other in order to address a existing mammalian condition. In other embodiments, specific combinations of enzymes can be included in the enzyme portion to address a specific mammalian condition. By way of non-limiting example, if a desired blood pH of a mammal is within the range of 7.3 to 7.5 is desired, and a blood pH measured from a mammal is higher than 7.5, a product according to the present invention can include an enzyme portion including enzymes which facilitate digestion, such as one or more of protease, lactase; amylase; macerate and lipase and a primary substance can include magnesium. Other examples can include a pH level of urine and pH levels of saliva and a combination of any or all these pH measurements.

In another aspect of the present invention, although the discussion above has generally been directed to capsules meant for ingestion into the human body, the scope of the present invention includes any application where an advantage can be gained from packaging an enzyme in isolation from a primary substance. In addition, some embodiments may include packaging two or more enzymes in a single package, wherein the enzymes are maintained in isolation until a predetermined condition is met which breaks the integrity of the package.

Some embodiments can therefore include packaging in isolation, an enzyme for breaking down crude oil in one package with an organism which can use broken crude oil chains as an energy source. It may be advantageous to isolate the enzyme from the organism until they are both exposed to crude oil which needs to be broken down, such as in the case of an oil spill, or an environment in proximity to the crude oil. The enzyme and organism can then be presented to the crude oil at the same time and place and in predetermined ratios.

Still other embodiments can include capsules designed to degrade in conditions found in a grease trap to release enzymes on location to clean the grease trap. Another embodiment can include capsules designed to facilitate conversion of sludge, such as grease trap sludge, into bio-fuel, including, for example bio-diesel fuel. The sludge may include yellow or brown sludge.

### CONCLUSION

A number of embodiments of the present invention have been described. Nevertheless, it will be understood that various modifications may be made without departing from the spirit and scope of the invention. For example, various methods or equipment may be used to implement the process steps described herein or to create a combination package according to the inventive concepts provided above and further described in the claims. In addition, various combinations of enzymes and substances may require particular packaging materials. Accordingly, other embodiments are within the scope of the following claims.

1. A package for an enzyme portion and a primary substance portion, the package comprising:
an inner boundary defining an inner containment area, said inner boundary comprising a material effective at isolating the enzyme portion from the primary substance portion while the integrity of the inner boundary is intact and the enzyme portion is placed on one side of the boundary and the primary substance is placed on another side of the inner boundary; and
an outer boundary defining an outer containment area, said outer boundary comprising a material effective at containing the enzyme portion, the inner boundary and the primary substance portion.

2. The package of claim 1 wherein the inner boundary comprises a softgel suitable to encase a predetermined volume of the primary substance and isolate the primary substance from the enzyme portion.

3. The package of claim 2 wherein the outer boundary comprises a hard capsule of sufficient size to contain the softgel and a predetermined amount of the enzyme portion.

4. The package of claim 3 wherein the softgel is suitable to isolate the enzyme portion from the primary substance until a predetermined set of conditions is encountered by the softgel.

5. The package of claim 4 wherein the predetermined set of conditions comprise conditions equivalent to ingestion in a human stomach.

6. The package of claim 4 wherein the predetermined set of conditions comprise conditions equivalent to ingestion in a human small intestine.

7. The package of claim 4 wherein the inner boundary maintains isolation of the enzyme portion from the primary substance sufficient to prevent the enzyme portion from acting as a catalyst in a chemical reaction which changes the primary substance to one or more secondary substances.

8. The package of claim 4 wherein the enzyme portion comprises protease and the primary substance comprises an omega-3 oil.

9. The package of claim 4 wherein the enzyme portion comprises amylase and the primary substance comprises an omega-3 oil.

10. The package of claim 4 wherein the enzyme portion comprises lipase and the primary substance comprises an omega-3 in a carrier oil.

11. The package of claim 4 wherein the enzyme portion comprises lipase and the primary substance comprises an omega-6 in a carrier oil.

12. The package of claim 4 wherein the primary substance comprises a carrier oil comprising one or more of: safflower oil and flax seed oil.

13. The package of claim 4 wherein the primary substance comprises one or more fatty acids presented as triglycerides, and the enzyme portion comprises a lipase.

14. A method of packaging an enzyme portion and a substance capable of being hydrolyzed by the enzyme in a package unit, the method comprising the steps of:
containing one of: the enzyme portion and the substance capable of being hydrolyzed by the enzyme portion within an inner boundary;
placing the one of: the enzyme portion and the substance capable of being hydrolyzed by the enzyme portion, that is not contained within the inner boundary, external to the inner boundary and internal to a outer boundary.

15. The method of claim 14 wherein the inner boundary comprises a softgel encapsulation.

16. The method of claim 15 wherein the outer boundary comprises a hard capsule of sufficient size to contain the softgel and a predetermined amount of the enzyme portion.

17. The method of claim 15 wherein the hard capsule comprises a gelatin.

18. A method of packaging an enzyme portion and a substance capable of being hydrolyzed by the enzyme in a package unit, the method comprising the steps of:
containing one of: the enzyme portion and the substance capable of being hydrolyzed by the enzyme portion within micro-encapsulation;
placing the one of: the enzyme portion and the substance capable of being hydrolyzed by the enzyme that is microencapsulated and the one of the one of:
the enzyme portion and the substance capable of being hydrolyzed by the enzyme portion that is not encapsulated within an outer boundary.

19. The method of claim 18 wherein the outer boundary comprises a hard gelatin capsule.

20. The method of claim 18 wherein both the enzyme portion and the substance capable of being hydrolyzed by the enzyme portion are microencapsulated and contained within the outer boundary.
